(19) United States
(12) Patent Application Publication
(10) Pub. No.: US 2013/0157262 A1
(43) Pub. Date: Jun. 20, 2013

(54) Method for Selecting Antibody-Producing Cell Line, and Kit Thereof

(71) Applicants: Eun Gyo Lee, Daejeon (KR); Hong Weon Lee, Daejeon (KR); Yeon Gu Kim, Daejeon (KR); Joon Ki Jung, Daejeon (KR); Jung Oh Ahn, Daejeon (KR); Chun Sug Kim, Daejeon (KR); Hyeok Won Lee, Daejeon (KR); Joo Hwan Lee, Daejeon (KR); Hye Lym Lee, Daejeon (KR)

(72) Inventors: Eun Gyo Lee, Daejeon (KR); Hong Weon Lee, Daejeon (KR); Yeon Gu Kim, Daejeon (KR); Joon Ki Jung, Daejeon (KR); Jung Oh Ahn, Daejeon (KR); Chun Sug Kim, Daejeon (KR); Hyeok Won Lee, Daejeon (KR); Joo Hwan Lee, Daejeon (KR); Hye Lym Lee, Daejeon (KR)

(73) Assignee: Korea Research Institute of Bioscience and Biotechnology, Daejeon (KR)

(21) Appl. No.: 13/643,952

(22) PCT Filed: Apr. 29, 2011
(86) PCT No.: PCT/KR2011/003189

§ 371 Date: Feb. 25, 2013

(30) Foreign Application Priority Data
Apr. 29, 2010 (KR) 10-2010-0040248
Apr. 27, 2011 (KR) 10-2011-0039550

(51) Int. Cl. C12Q 1/02

(52) U.S. Cl. CPC C12Q 1/025
USPC 435/6.1

(57) ABSTRACT

Provided is a method for selecting antibody-producing cell lines using a split fluorescent protein, and a kit for selecting antibody-producing cell lines. By using the split fluorescent protein to select the antibody-producing cell lines, the antibody-producing cell lines can be easily detected by observing whether a single fluorescent color derived from reassembly of the split fluorescent proteins is expressed, which leads in a drastic reduction in selection time and cost required to select highly productive antibody-producing cell line.

### BACKGROUND

1. Field of the Invention

The present invention relates to a method for selecting antibody-producing cell lines using a split fluorescent protein, and a kit for selecting antibody-producing cell lines.

2. Discussion of Related Art

Selection of cell lines having high productivity is an important step in production of antibodies for treatment using animal cell lines. Conventional methods for selecting animal cell lines having high productivity include a limiting dilution method, gel microdrop technology, and an automated machine method. Although the limiting dilution method is currently the most widely used due to its simple and low-cost advantage for selecting animal cell lines which produce antibodies for treatment, it is inefficient compared with other methods, and is not suitable for high throughput screening (HTS). On the other hand, although the gel microdrop technology and the automated machine method have excellent advantages in HTS, these methods are not widely used due to the complexity of the methods themselves as well as high cost. In detail, the gel microdrop technology requires intricate processes and has limits in low efficiency of gel formation etc. The automated machine method requires a relatively high cost, which is a big problem in use.

As such, development of a new system which enables selection of highly productive antibody-producing cell lines with a simple, efficient and low-cost method is needed.

### SUMMARY OF THE INVENTION

The present invention is directed to providing a method for easily selecting antibody-producing cell lines using reassembly force of a split fluorescent protein, and a kit for selecting the cell lines.

One aspect of the present invention provides a method for selecting antibody-producing cell lines, including transfecting a first expression vector and a second expression vector into cells, wherein the first expression vector includes a sequence which encodes a first fragment of a fluorescent protein and a sequence which encodes a heavy chain of an antibody, and the second expression vector includes a sequence which encodes a second fragment of the fluorescent protein and a sequence which encodes a light chain of the antibody; and selecting the antibody-producing cell lines by confirming a fluorescence derived from reassembly between the first fragment and the second fragment of the fluorescent protein.

Another aspect of the present invention provides a kit for selecting antibody-producing cell lines, including a first expression vector including a sequence which encodes a first fragment of a fluorescent protein and an insertion site into which a sequence which encodes a heavy chain of an antibody may be introduced; and a second expression vector including a sequence which encodes a second fragment of the fluorescent protein and an insertion site into which a sequence which encodes a light chain of the antibody may be introduced.

By using the split fluorescent protein to select the antibody-producing cell lines according to the present invention, the antibody-producing cell lines can be easily detected by observing whether a single fluorescent color derived from reassembly of the split fluorescent proteins is expressed, which leads in a drastic reduction in selection time and cost required to select highly productive antibody-producing cell lines.

### BRIEF DESCRIPTION OF THE DRAWINGS

The above and other objects, features and advantages of the present invention will become more apparent to those of ordinary skill in the art by describing in detail exemplary embodiments thereof with reference to the attached drawings, in which:

FIG. 1 is a schematic diagram showing that green fluorescence derived from a reassembled green fluorescent protein (GFP) which is formed by expression of each of an N-terminal fragment and a C-terminal fragment of the GFP may be used as a marker indicating a formation of an antibody by simultaneous expression of a heavy chain and a light chain structure of the antibody, and that the selection of antibody-producing animal cell lines is possible using the same.

FIG. 2 is a drawing showing an observation result of a formation of a reassembled GFP by simultaneous expression of the expression vectors pcDNA-NGFP and pcDNA-CGFP through confocal microscope.

FIG. 3 is a schematic diagram illustrating the structures of pNGFP-Light, a vector which simultaneously expresses an N-terminal fragment of a GFP and light chain structure of an antibody, and of pCGFP-Heavy, a vector which simultaneously expresses a C-terminal fragment of the GFP and heavy chain structure of the antibody, according to the present invention.

FIG. 4A is a drawing showing an observation result of a formation of reassembled GFP by simultaneous expression of the expression vectors pNGFP-Light and pCGFP-Heavy through a confocal microscope according to the present invention.

FIG. 4B is a drawing showing a measurement result of an amount of an antibody produced by simultaneous expression of the expression vectors pNGFP-Light and pCGFP-Heavy through enzyme immunoassay according to the present invention.

FIG. 5 is a graph showing a ratio of GFP expression cells and specific antibody productivity of a primary separation pool and a secondary separation pool established by separating individual cells (upper 1%) which highly express a GFP from CHO cell lines (non-separation pool) which simultaneously express the expression vectors pNGFP-Light and pCGFP-Heavy using fluorescence-activated cell sorter according to the present invention.

FIG. 6 is a graph indicating a co-relationship between a degree of GFP expression and specific antibody productivity for 30 individual cell lines established according to the present invention.

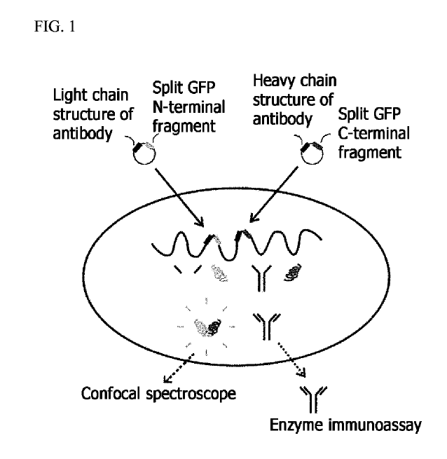

### DETAILED DESCRIPTION OF EXEMPLARY EMBODIMENTS

The present invention provides a method for selecting antibody-producing cell lines, including transfecting a first expression vector and a second expression vector into cells, wherein the first expression vector includes a sequence which encodes a first fragment of a fluorescent protein and a sequence which encodes a heavy chain of an antibody, and the second expression vector includes a sequence which encodes a second fragment of the fluorescent protein and a sequence which encodes a light chain of the antibody; and selecting the antibody-producing cell lines by confirming fluorescence derived from reassembly between the first fragment and the second fragment of the fluorescent protein.

In one embodiment of the present invention, the fluorescent protein may be, for example, a green fluorescent protein (GFP), a red fluorescent protein (RFP), a blue fluorescent protein (BFP), a yellow fluorescent protein (YFP), a cyan fluorescent protein (CFP) or an enhanced fluorescent protein (EFP), but the present invention is not limited thereto, and any fluorescent proteins may be used as long as they shows fluorescence by stimulus of light.

The fluorescent protein fragment according to the present invention denotes a split fluorescent protein, and the split fluorescent protein indicates a fragment of the fluorescent protein which loses its capability to emit fluorescence when split into a plurality of fragments, but recovers its capability to emit fluorescence when the plurality of fragments are reassembled.

In the present invention, the term “reassembly” denotes an assembly of the fragments of the fluorescent protein that have lost their fluorescence-emitting capability so as to construct the fluorescent protein whose fluorescence-emitting capability is recovered.

As shown through Examples 1 and 2 below, an experiment was performed to determine the recovery of the fluorescence-emitting capability of the fluorescent protein reassembled using fluorescent protein fragments of a GFP, which is a representative example of fluorescent proteins. As a result, it is confirmed that the fluorescence-remitting capability of the fluorescent protein is recovered as the fluorescent protein fragments were reassembled by transfecting the two vectors, both of which respectively expresses a first fragment and a second fragment of the fluorescent protein which lost their fluorescence-emitting capability, into the same animal cell and expressing the first fragment and the second fragment.

The first expression vector including a sequence which encodes a first fragment of the fluorescent protein and a sequence which encodes a heavy chain of an antibody and the second expression vector including a sequence which encodes a second fragment of the fluorescent protein and a sequence which encodes a light chain of the antibody according to the present invention simultaneously express the first fragment of the fluorescent protein and the heavy chain of the antibody, and the second fragment of the fluorescent protein and the light chain of the antibody, respectively. As such, the first and second expression vectors have a correlation between the expression amount of each fragment of the fluorescent protein and of the heavy chain or the light chain of the antibody within one vector. Accordingly, when the first expression vector and the second expression vector are transfected into the same cells, the heavy chain and the light chain of the antibody are assembled, and the ratio of formation of the antibody protein is proportional to the ratio of reassembly of the first fragment and the second fragment of the fluorescent protein so as to show fluorescence. As such, an amount of fluorescence shown by the reassembled fluorescent protein may be used as a marker indicating a production amount of the antibody. In other words, highly productive antibody-producing cell lines which emit large amounts of fluorescence may be easily selected by confirming the amount of fluorescence derived from reassembly of the first fragment and the second fragment of the fluorescent protein. Herein, the selection of the cell lines may be performed using any methods known in the related art, for example, fluorescence activated cell sorting (FACS) method.

In a specific embodiment of the present invention, one of the first fragment and the second fragment of the fluorescent protein may be a C-terminal fragment of the fluorescent protein, and the other may be an N-terminal fragment of the fluorescent protein. In other words, if the first fragment of the fluorescent protein is a C-terminal fragment of the fluorescent protein, the second fragment of the fluorescent protein is an N-terminal fragment of the fluorescent protein, and if the first fragment of the fluorescent protein is an N-terminal of the fluorescent protein, the second fragment of the fluorescent protein is a C-terminal fragment of the fluorescent protein.

Also, a cleavage site in a full-length fluorescent protein for producing the first fragment and second fragment of the fluorescent protein may be appropriately selected by a person skilled in the art as long as fluorophores are conserved in the fragments of the fluorescent protein, so that the fluorescence-emitting capability may be recovered by reassembly of the fragments, and may be a site in which some sequences in the full-length fluorescent protein sequence are inserted or missing. For example, when a GFP is used as the fluorescent protein, the cleavage site for producing the first fragment and the second fragment of the GFP may be between 157^{th} and 158^{th} amino acid residues of the full-length GFP (see U.S. Pat. No. 6,780,599), but the present invention is not limited thereto. In Examples of the present invention, a sequence set forth in SEQ ID NO: 5 was used as a sequence which encodes the first fragment of the fluorescent protein, and a sequence set forth in SEQ ID NO: 11 was used as a sequence to encode the second fragment of the fluorescent protein.

In another embodiment of the present invention, the first expression vector further includes a sequence which encodes a first linker peptide conjugated to the sequence which encodes the first fragment of the fluorescent protein, and the second expression vector further includes a sequence which encodes a second linker peptide conjugated to the sequence which encodes the second fragment of the fluorescent protein. In this sense, the first linker peptide and the second linker peptide may be constructed so as to be conjugated to each other.

In the present invention, the term “expression vector” denotes a DNA construct including an exogenous DNA sequence operably conjugated to an appropriate regulatory sequence which is able to express DNA in an appropriate host. The expression vector of the present invention may be constructed as a representative vector for expression. Preferably, the expression vector of the present invention is a vector for expressing a recombinant peptide or protein. In addition, the expression vector of the present invention may be constructed with a prokaryotic cell or a eukaryotic cell as a host cell. The recombinant expression vector of the present invention may be, for instance, a bacteriophage vector, a cosmid vector, or a yeast artificial chromosome (YAC) vector. According to the purpose of the present invention, a plasmid vector may be preferably used. The representative plasmid vector which may be used for such purpose has (a) a replication origin which enables efficient replication so as to include hundreds of plasmid vectors per host cell, (b) a marker gene which enables a selection of the host cell which was transformed with the plasmid vector, and (c) a structure including a restriction enzyme cleavage site at which an exogenous DNA fragment may be inserted. The vector and exogenous DNA may be easily ligated using the synthetic oligonucleotide adaptor or linker according to the conventional method, even in the absence of an appropriate restriction enzyme cleavage site. The expression vector used in the present invention may be constructed by various methods known in the related art.

In the first expression vector according to the present invention, a sequence which encodes the first fragment of the fluorescent protein; a sequence which encodes the first linker peptide conjugated to the sequence; and a sequence which encodes the heavy chain of the antibody are operably linked, and also, in the second expression vector, a sequence which encodes the second fragment of the fluorescent protein; a sequence which encodes the second linker peptide conjugated to the sequence; and a sequence which encodes a light chain of the antibody are operably linked.

Being “operably linked” denotes a functional combination between a regulatory sequence for nucleic acid expression (e.g. a promoter, a signal sequence, or an array of a conjugation site for a transcription factor) and another nucleic acid sequence (e.g. a sequence which encodes a fragment of the fluorescent protein), wherein the regulatory sequence regulates a transcription and/or translation process of the other nucleic acid sequences.

The first linker peptide and the second linker peptide are constructed so as to be conjugated to each other. The sequences which encode the first linker peptide and the second linker peptide are directly conjugated to the sequences which encode the first fragment and the second fragment of the fluorescent protein, respectively, and as such, upon the expression of the protein, the first linker peptide and the first fragment of the fluorescent protein, and the second linker peptide and the second fragment of the fluorescent protein are expressed into a fusion protein. Accordingly, when the first linker peptide and the second linker peptide are conjugated to each other, the first fragment and the second fragment of the fluorescent protein come close and are reassembled to recover the property of emitting fluorescence. The linker peptide may be, for example, a leucine zipper as disclosed in U.S. Pat. No. 6,780,599, or an EF1/EF2 peptide as disclosed in Chen, N. et al. J. Biotechnol., 2009, 142, 205-213 and Lindman, S. et al. Protein Sci., 2009, 18, 1221-1229, but the present invention is not limited thereto. In Examples of the present invention, an EF1 sequence was used as the first linker peptide sequence, and an EF2 sequence was used as the second linker peptide sequence.

The construction of the vector to simultaneously express a fluorescent protein fragment and the heavy chain or light chain of the antibody within one vector according to the present invention may be appropriately embodied by a person skilled in the art, and for example, a bicistronic expression vector or an internal ribosome entry site (IRES) inside a pIRES expression vector may be used, but the present invention is not limited thereto.

In a further embodiment of the present invention, the method may further include IRES sequences between the sequence which encodes the first fragment of the fluorescent protein of the first expression vector and an insertion site into which the sequence which encodes the heavy chain of the antibody are introduced; and between the sequence which encodes the second fragment of the fluorescent protein of the second expression vector and an insertion site into which the sequence which encodes the light chain of the antibody are introduced. When using an IRES, the expression of fluorescent protein fragments and the heavy chain or light chain of the antibody may be regulated at different ratios within each vector, and thus, use of the IRES is preferred.

The antibody-producing cell lines which may be used in the present invention are usually animal cell lines, but the present invention is not particularly limited thereto, and may be E. coli, yeast or plant cell lines. When the antibody-producing cell lines are animal cell lines, the animal cells may be HEK293, COS7, HeLa, or CHO cells, but the present invention is not limited thereto.

In another embodiment of the present invention, the method for selecting antibody-producing cell lines may further include confirming a production of the antibody by the antibody-producing cell lines selected by the above-described method. This step is an additional step of verifying a correlation between the amount of fluorescence derived from reassembly of the first fragment and the second fragment of the fluorescent protein and the production amount of the antibody derived from assembly of the heavy chain and light chain of the antibody. Through this step, the actual antibody-producing capability of the antibody-producing cell lines which are primarily selected by the amount of the produced fluorescence may be secondarily verified to accurately select the antibody-producing cell lines. As the method of confirming a production of the antibody by the antibody-producing cell lines, any known methods in the art may be used, and for example, enzyme immunoassay may be used.

Moreover, the present invention provides a kit for selecting antibody-producing cell lines, including a first expression vector including a sequence which encodes a first fragment of a fluorescent protein and an insertion site into which a sequence which encodes a heavy chain of an antibody may be introduced; and a second expression vector including a sequence which encodes a second fragment of the fluorescent protein and an insertion site into which a sequence which encodes a light chain of the antibody may be introduced.

The expression “an insertion site into which a sequence which encodes a heavy chain of an antibody may be introduced” in the first expression vector; or the expression “an insertion site into which a sequence which encodes a light chain of the antibody may be introduced” in the second expression vector denote a region including the restriction enzyme cleavage site at which an exogenous DNA fragment which encodes the heavy chain or the light chain of a target antibody may be inserted. If there is no appropriate restriction enzyme cleavage site, they denote the region consisting of the structure in which the vector and exogenous DNA can be easily ligated using the synthetic oligonucleotide adaptor or linker according to the conventional method. The detailed construction of the insertion site into which such an exogenous DNA fragment may be introduced may be appropriately performed by a person skilled in the art.

By using the kit for selecting antibody-producing cell lines according to the present invention including the first expression vector and the second expression vector, each including the insertion site into which the sequence which encodes the heavy chain or the light chain of the antibody may be introduced, the cells which produce the target antibody may be easily selected by introducing the sequences which encode the heavy chain and the light chain of the target antibody to the respective entry regions, and by producing the expression vector to select the cell lines which produce the target antibody in an easy and fast manner.

The kit for selecting antibody-producing cell lines according to the present invention may include, in addition to the first expression vector and the second expression vector, any biological or chemical agents, or manuals needed to select antibody-producing cell lines, including a restriction enzyme and a primer to insert the target antibody gene into the expression vectors, and a transfection agent and a parental cell to introduce the prepared expression vector into the cells. Such other components of the kit may be appropriately selected by a person skilled in the art.

In one embodiment according to the present invention, the fluorescent protein may be, for example, a GFP, an RFP, a BFP, a YFP, a CFP or an EFP.

In a specific embodiment of the present invention, one of the first fragment and the second fragment of the fluorescent protein may be a C-terminal fragment of the fluorescent protein, and the other may be an N-terminal fragment of the fluorescent protein.

In another embodiment of the present invention, the first expression vector further includes a sequence which encodes a first linker peptide conjugated to the sequence which encodes the first fragment of the fluorescent protein, and the second expression vector further includes a sequence which encodes a second linker peptide conjugated to the sequence which encodes the second fragment of the fluorescent protein. In this sense, the first linker peptide and the second linker peptide may be constructed so as to be conjugated to each other.

In a further embodiment of the present invention, the kit may further include an IRES sequence between the sequence which encodes the first fragment of the fluorescent protein of the first expression vector and an insertion site into which the sequence which encodes the heavy chain of the antibody are introduced; and between the sequence which encodes the second fragment of the fluorescent protein of the second expression vector and an insertion site into which the sequence which encodes the light chain of the antibody are introduced.

In the kit for selection according to said embodiments, the characteristics of the first expression vector and the second expression vector included in the kit for selection include all the characteristics of the first expression vector and the second expression vector used in the method of selecting antibody-producing cell lines as described above, except that they include the insertion site into which the sequences which encode the heavy chain or the light chain of any antibody are introduced instead of including sequences which encode the heavy chain or the light chain of a certain antibody, and the further details are omitted to avoid repeated explanations.

Hereinafter, exemplary embodiments of the present invention will be described in detail. However, the present invention is not limited to the embodiments disclosed below, but may be implemented in various forms. The following embodiments of the present invention are provided for better understanding of the present invention by those of ordinary skill in the art.

### EXAMPLES

### Example 1

### Preparation of the Expression Vectors, pcDNA-NGFP and pcDNA-CGFP

In order to produce the vectors which express an N-terminal fragment and a C-terminal fragment of a GFP in animal cells, the fluorescent protein part of each terminal fragment was obtained using pEGFP-C1 (available from Clontech) as a template, and the linker protein of each terminal fragment was synthesized with DNA at BIONEER. In order to clone the N-terminal fragment of the GFP, a total of 25 cycles of PCR reactions were performed to perform amplification under the conditions of 1 min 30 s at 92° C., 1 min 30 s at 55° C. and 2 min at 72° C. using an F1 primer (SEQ ID NO: 1) and an R1 primer (SEQ ID NO: 2) and pEGFP-C1 as the template. The amplified N-terminal fragment DNA was named Seq-1 DNA (SEQ ID NO: 5). The portion corresponding to Seq-2 DNA (SEQ ID NO: 6) to synthesize a linker peptide consisting of an EF1 sequence and a restriction enzyme site was synthesized at BIONEER. A total of 35 cycles of PCR reactions were performed to perform amplification under the conditions of 1 min 30 s at 92° C., 2 min at 50° C. and 2 min at 72° C. using an F2 primer (SEQ ID NO: 3) and an R2 primer (SEQ ID NO: 4) and an assembly of Seq-1 DNA and Seq-2 DNA, both of which are contained at the same amount, as the template. After digestion with the restriction enzymes HindIII and NotI, it was inserted into the expression vector, pcDNA3.1/Zeo (available from Invitrogen), which was digestion with the same enzymes, and named as the expression vector pcDNA-NGFP.

In order to clone a C-terminal fragment of the GFP, a total of 25 cycles of PCR reactions were performed to amplify under the conditions of 1 min 30 s at 92° C., 1 min 30 s at 55° C. and 2 min at 72° C. using an F3 primer (SEQ ID NO: 7) and an R3 primer (SEQ ID NO: 8) and pEGFP-C1 as the template. The amplified C-terminal fragment DNA was named Seq-3 DNA (SEQ ID NO: 11). The portion corresponding to Seq-4 DNA (SEQ ID NO: 12) to synthesize a linker peptide consisted of an EF2 sequence and a restriction enzyme site was synthesized at BIONEER. A total of 35 cycles of PCR reactions were performed to perform amplification under the conditions of 1 min 30 s at 92° C., 2 min at 50° C. and 2 min at 72° C. using an F4 primer (SEQ ID NO: 9) and an R4 primer (SEQ ID NO: 10) and an assembly of Seq-3 DNA and Seq-4 DNA, both of which are contained at the same amount, as the template. After digestion with the restriction enzymes HindIII and NotI, it was inserted into the expression vector, pcDNA3.1/Zeo, which was digested with the same enzymes, and named as the expression vector pcDNA-CGFP.

TABLE 1

| Sequence | Sequence | |
|---|---|---|
| No. | name | Sequence |
| 1 | F1 | 5′-ATGGTGAGCAAGGGCGAG-3′ |
| | Primer | |
| 2 | R1 | 5′-CTGCTTGTCGGCCATGAT-3′ |
| | Primer | |
| 3 | F2 | 5′- |
| | Primer | GCGAAGCTTGCCACCATGGTGAGCAAGGGCGAGGAGC- |
| | | 3′ |
| 4 | R2 | 5′-GCGGCGGCCGCTCATGGACCCTTCAGCAAACTGGG-3′ |
| | Primer | |
| 5 | Seq-1 | 5′- |
| | DNA | ATGGTGAGCAAGGGCGAGGAGCTGTTCACCGGGGTGGTGCCCATCC |
| | | TGGTCGAGCTGGACGGCGACGTAAACGGCCACAAGTTCAGCGTGTC |
| | | CGGCGAGGGCGAGGGCGATGCCACCTACGGCAAGCTGACCCTGAA |
| | | GTTCATCTGCACCACCGGCAAGCTGCCCGTGCCCTGGCCCACCCTCG |
| | | TGACCACCCTGACCTACGGCGTGCAGTGCTTCAGCCGCTACCCCGA |
| | | CCACATGAAGCAGCACGACTTCTTCAAGTCCGCCATGCCCGAAGGC |
| | | TACGTCCAGGAGCGCACCATCTTCTTCAAGGACGACGGCAACTACA |
| | | AGACCCGCGCCGAGGTGAAGTTCGAGGGCGACACCCTGGTGAACC |
| | | GCATCGAGCTGAAGGGCATCGACTTCAAGGAGGACGGCAACATCCT |
| | | GGGGCACAAGCTGGAGTACAACTACAACAGCCACAACGTCTATATC |
| | | ATGGCCGACAAGCAG-3′ |
| 6 | Seq-2 | 5′- |
| | DNA | CTACAACAGCCACAACGTCTATATCATGGCCGACAAGC |
| | | AGGGTGGCTCTGGCTCTGGCTCGAGCAAGTCTCCAGAA |
| | | GAACTGAAGGGCATTTTCGAAAAATATGCAGCCAAAGA |
| | | AGGTGATCCAAACCAACTGTCCAAGGAGGAGCTGAAGC |
| | | TACTGCTTCAGACGGAATTCCCCAGTTTGCTGAAGGGTC |
| | | CATGA-3′ |
| 7 | F3 | 5′-AAGAACGGCATCAAGGTG-3′ |
| | Primer | |
| 8 | R3 | 5′-TCAGTACAGCTCGTCCAT-3′ |
| | Primer | |
| 9 | F4 | 5′- |
| | Primer | GCGAAGCTTGCCACCATGAGCACCCTCGATGAGCTTTTT |
| | | GAAG-3′ |
| 10 | R 4 | 5′-GCGGCGGCCGCTCAGTACAGCTCGTCCATGCCG-3′ |
| | Primer | |
| 11 | Seq-3 | 5′- |
| | DNA | AAGAACGGCATCAAGGTGAACTTCAAGATCCGCCACAA |
| | | CATCGAGGACGGCAGCGTGCAGCTCGCCGACCACTACC |
| | | AGCAGAACACCCCCATCGGCGACGGCCCCGTGCTGCTG |
| | | CCCGACAACCACTACCTGAGCACCCAGTCCGCCCTGAG |
| | | CAAAGACCCCAACGAGAAGCGCGATCACATGGTCCTGC |
| | | TGGAGTTCGTGACCGCCGCCGGGATCACTCTCGGCATG |
| | | GACGAGCTGTACTGA-3′ |
| 12 | Seq-4 | 5′- |
| | DNA | AGCACCCTCGATGAGCTTTTTGAAGAACTAGACAAGAA |
| | | TGGAGATGGAGAAGTTAGTTTCGAAGAATTCCAGGTGT |
| | | TGGTGAAAAAGATATCCCAGACGTCGGGTGGAAGCGGT |
| | | AAGAACGGCATCAAGGTGAACTTCAAGATCCGCCACAA |
| | | CA-3′ |

### Example 2

### Formation of Reassembled Fluorescent Protein Through Simultaneous Expression of the Expression Vectors pcDNA-NGFP and pcDNA-CGFP, and Observation of Fluorescence Through Confocal Microscope

In order to confirm the production of fluorescence derived from a formation of the GFP in cells reassembled by the vectors expressing the N-terminal fragment and the C-terminal fragment of the GFP prepared in Example 1, pcDNA-NGFP and pcDNA-CGFP were transfected into HEK293 cells. The HEK293 cells were subcultured in the animal cell medium DMEM (available from HyClone) further supplemented with 10% FBS (available from Invitrogen) and 4 mM glutamine (available from Sigma). A coverslip to which the HEK293 cells could be attached was attached to the bottom of a 12-well plate (available from Nunc), and the HEK293 cells were cultured for 12 hours. Thereafter, 0.5 mg of a vector was transfected to the cells in a 1.5 mL transfection solution (available from Stratagene). As a control, the vectors, pEGFP-C1 and pcDNA3.1/Zeo, which expressed the GFP were transfected to HEK293 cells. As an experimental group, the vectors pcDNA-NGFP and pcDNA-CGFP, which expressed the N-terminal fragment and the C-terminal fragment of the fluorescent protein, were individually transfected. Finally, the mixture of pcDNA-NGFP and pcDNA-CGFP was transfected. The transfected cells were further cultured for 24 hours at 30° C. In order to observe fluorescence from the reassembled GFP under the confocal microscope, the medium was removed from the 12-well plate. The transfected HEK293 cells on the coverslip attached to the bottom of the 12-well plate were washed once with a PBS solution, and then were immobilized for 10 min with a PBS solution including 2% paraformaldehyde. After washing the immobilized cells twice with a PBS solution, they were treated with a mounting solution including 4,6-diamidino-2-phenylindole (DAPI) for nuclear staining. Finally, the coverslip to which the transfected HEK293 cells were attached was observed under the confocal microscope (available from Zeiss).

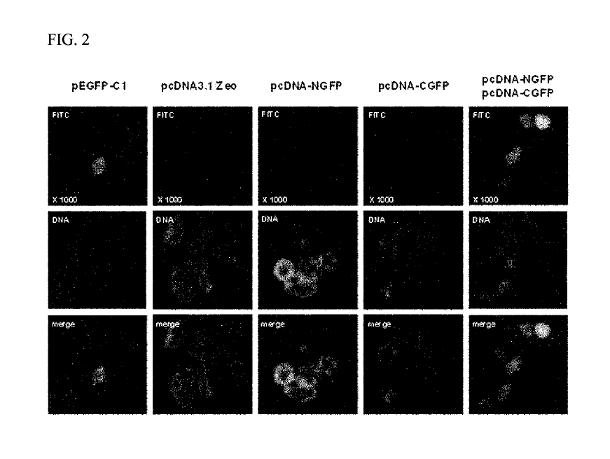

As a result, as shown in FIG. 2, it was confirmed that the green color of the GFP was observed when the mixture of pcDNA-NGFP and pcDNA-CGFP was transfected.

### Example 3

### Preparation of Expression Vectors pNGFP-Light and pCGFP-Heavy

In order to insert the light chain structure of the antibody in the position of the multi cloning site (MCS)-A of pIRES (available from Clontech) which is the vector that simultaneously expresses two genes, a total of 25 cycles of PCR reactions were performed to perform amplification under the conditions of 1 min 30 s at 92° C., 1 min 30 s at 55° C. and 1 min at 72° C. using an F5 primer (SEQ ID NO: 13) and an R5 primer (SEQ ID NO: 14) and cDNA, which corresponded to the light chain structure of the model antibody kindly provided by PhaonABcine, as the template. The amplified DNA was digested with the restriction enzymes NheI and MluI, and then was inserted into the expression vector pIRES which was digested with the same enzymes, and named as the expression vector pIRES-Light.

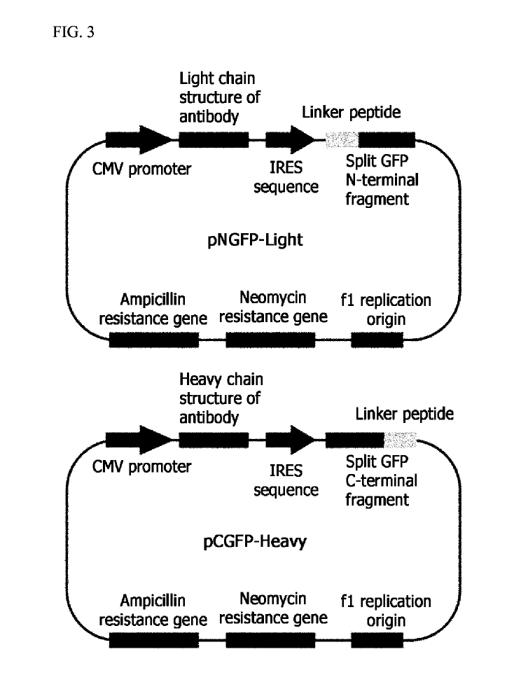

For the N-terminal fragment of the fluorescent protein, a total of 25 cycles of PCR reactions were performed to perform amplification under the conditions of 1 min 30 s at 92° C., 1 min 30 s at 55° C. and 1 min at 72° C. using an F6 primer (SEQ ID NO: 15) and an R6 primer (SEQ ID NO: 16) and pcDNA-NGFP prepared in Example 1 as the template. The amplified DNA was digested with the restriction enzymes, XbaI and SalI, and then was inserted into the expression vector MCS-B of pIRES-Light which was digested with the same enzymes, and named as the expression vector pNGFP-Light (FIG. 3).

In order to insert the heavy chain structure of the antibody in the position of expression vector MCS-A of pIRES, a total of 25 cycles of PCR reactions were performed to perform amplification under the conditions of 1 min 30 s at 92° C., 1 min 30 s at 55° C. and 2 min at 72° C. using an F7 primer (SEQ ID NO: 17) and an R7 primer (SEQ ID NO: 18) and cDNA, which corresponded to the heavy chain structure of the model antibody kindly provided by PhaonABcine, as the template. The amplified DNA was digested with the restriction enzyme EcoRI, and then was inserted to the expression vector pIRES which was digested with the same enzyme, and named as the expression vector pIRES-Heavy.

For the C-terminal fragment of the fluorescent protein, a total of 25 cycles of PCR reactions were performed to perform amplification under the conditions of 1 min 30 s at 92° C., 1 min 30 s at 55° C. and 1 min at 72° C. using an F8 primer (SEQ ID NO: 19) and an R8 primer (SEQ ID NO: 20) and pcDNA-CGFP prepared in Example 1 as the template. The amplified DNA was digested with the restriction enzymes XbaI and SalI, and then was inserted to the expression vector MCS-B of pIRES-Heavy which was digested with the same enzymes, and named as the expression vector pCGFP-Heavy (FIG. 3).

TABLE 2

| Sequence | Sequence | |
|---|---|---|
| No. | name | Sequence |
| 13 | F5 Primer | 5′-CTAGCTAGCGCCACCATGGGATGGAGCTATATC-3′ |
| 14 | R5 Primer | 5′-CGACGCGTCTAACACTCTCCCCTGTT-3′ |
| 15 | F6 Primer | 5′-TGCTCTAGAGCCACCATGGTGAGCAAGGGCGAG- |
| | | 3′ |
| 16 | R6 Primer | 5′-GCGTGTCGACTCATGGACCCTTCAGCAA-3′ |
| 17 | F7 Primer | 5′-CCGGAATTCGCCACCATGGGATGGAGCTATATC-3′ |
| 18 | R7 Primer | 5′-CCGGAATTCTCATTTACCCGGAGACAG-3′ |
| 19 | F8 Primer | 5′-TGCTCTAGAGCCACCATGAGCACCCTCGATGAG-3′ |
| 20 | R8 Primer | 5′-GCGTGTCGACTCAGTACAGCTCGTCCAT-3′ |

### Example 4

### Verification of Fluorescence after Simultaneous Transfection of the Expression Vectors pNGFP-Light and pCGFP-Heavy, and Confirmation of Production of Antibody

In order to confirm whether the heavy chain structure and the light chain structure which are the two subunits of the antibody, and the N-terminal fragment and the C-terminal fragment of the fluorescent protein were produced from each of the expression vectors pNGFP-Light and pCGFP-Heavy prepared in Example 3, a mixture of the expression vectors pNGFP-Light and pCGFP-Heavy which were present at the same amount was transfected to HEK293 cells. The HEK293 cells were subcultured in the animal cell medium DMEM further supplemented with 10% FBS and 4 mM glutamine. After the transfection was conducted as explained in Example 3, the HEK293 cell culture medium was centrifuged (21,000×g, 20 min., 4° C.) for enzyme immunoassay, and then was stored at −80° C. The coverslip to which the transfected HEK293 cells were attached as in Example 2 was subjected to the immobilization step, and the fluorescence of the reassembled GFP was observed under the confocal microscope.

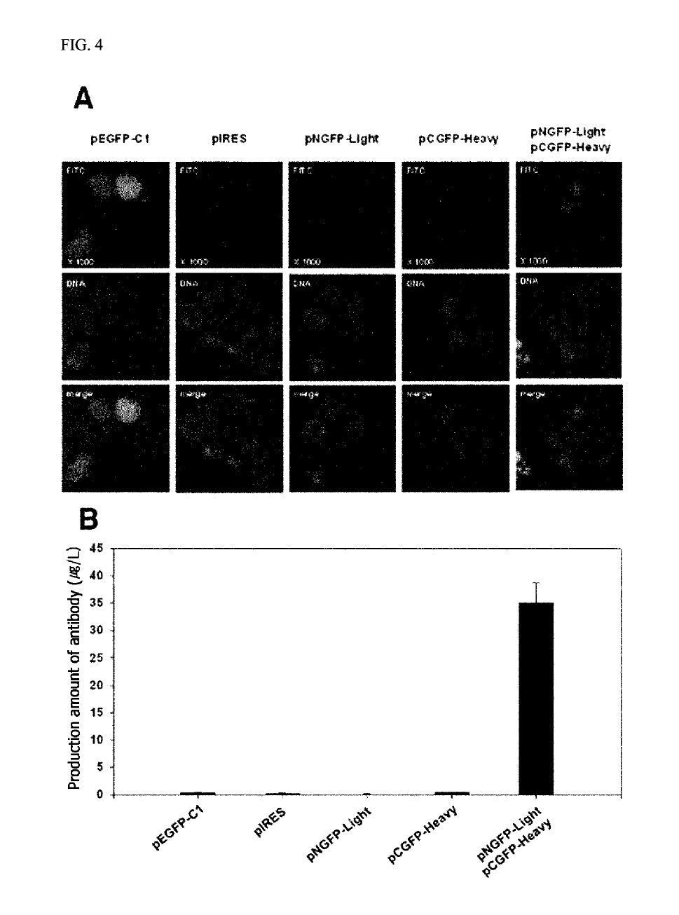

As a result, as shown in FIG. 4A, it was confirmed that the green color of the GFP was observed when the mixture of the expression vectors pNGFP-Light and pCGFP-Heavy was transfected.

In order to confirm whether the two subunits of the antibody, i.e. the heavy chain structure and the light chain structure, were assembled in the medium of HEK293 cells in which the mixture of the expression vectors pNGFP-Light and pCGFP-Heavy was transfected, enzyme immunoassay on the antibody was performed. After diluting goat anti-human IgG (available from Pierce) with a PBS solution including 2% skim milk (available from BD Biosciences), it was coated on a 96-well plate (available from Nunc) for 12 hours at 4° C. After washing the plate 3 times with a PBS solution, blocking was conducted for 1 hour at room temperature in a PBS solution including 2% skim milk. After washing 3 times with a PBST solution, which is a PBS solution including 0.05% Tween20, the culture medium and standard antibody were loaded on the plate and were reacted for 1 hour at 37° C. After the reaction, it was further washed 3 times with a PBST solution, and then reacted with alkaline phosphatase-conjugated goat anti-human IgG (available from Pierce) for 1 hour at 37° C. After washing 3 times with a PBST solution, a TMB solution (available from BD Biosciences) was finally added as a substrate. After the reaction was completed with sulfuric acid, measurement was performed at an absorbance of 450 nm using a multi-purpose plate reader (available from BioTek).

As a result, as shown in FIG. 4B, it was confirmed that the production of the antibody by the HEK293 cells was confirmed when the mixture of expression vectors pNGFP-Light and pCGFP-Heavy was transfected.

### Example 5

### Separation of Individual Cells which Expresses GFP with Fluorescence Activated Cell Sorting and Verification of Production of Antibody

In order to verify that the cells that showed green fluorescence due to the simultaneous expression of the N-terminal fragment and the C-terminal fragment of the GFP to form the reassembled GFP were antibody-producing animal cell lines, the expression vectors pNGFP-Light and pCGFP-Heavy prepared in Example 3 were transfected to Chinese hamster ovary-K1 (hereinafter “CHO-K1”) cells, and sub-cultured 3 times in the animal cell medium IMDM further supplemented with 10% FBS, 4 mM glutamine and 500 μg/mL G418. This was named as a non-separation pool. For the GFP reassembly, it was injected to a T-25 flask at a concentration of 0.7×10⁵ cells/mL, cultured for 3 days at 37° C., and then further cultured for 2 days at 30° C. Thereafter, the cells showing high GFP fluorescence, i.e. the upper 1% within the non-separation pool, were separated using a fluorescence-activated cell sorter, and then subcultured 3 times in the same medium, which was called “a primary separation pool.” With the same method, the cells showing high GFP fluorescence, i.e. the upper 1% within the primary separation pool, were separated using a fluorescence-activated cell sorter, and then subcultured 3 times in the same medium, which was called “a secondary separation pool.”

In order to measure a ratio of cells showing GFP fluorescence in the non-separation pool, the primary separation pool and the secondary separation pool, the culturing was performed for 3 days at 30° C., and verification was performed using a fluorescence-activated cell sorter. In addition, the cells of each pool were injected into a T-25 flask at a concentration of 0.7×10⁵ cells/mL, the total number of surviving cells was measured on the 2^{nd} day and 4^{th} day of culturing at 37° C., the produced antibody was measured using an enzyme immunoassay, and finally, specific antibody productivity was determined.

As a result, as shown in FIG. 5, the ratio of cells including the reassembled GFP among the total cell number of each pool increased after the separation using a fluorescence-activated cell sorter. Further, the higher specific productivity was measured in the pool in which the ratio of cells including the reassembled GFP increased.

### Example 6

### Verification of Effect of Separation Using Fluorescence-Activated Cell Sorter on Cell Line Selection

In order to verify an effect of the primary selection of cells using a fluorescence activated cell sorter on the selection of highly productive antibody-producing animal cell lines using the GFP as a marker, 116 clones were randomly selected from the non-separation pool and the secondary separation pool established in Example 5 using a limiting dilution method. After being injected into a 96-well plate at a concentration of 1 cell/3 wells, the clones were subcultured until they reached a T-25 flask. Thereafter, each clone was injected into a T-25 flask at a concentration of 3.0×10⁵ cells/mL. After culturing for 3 days at 37° C., the produced antibody was measured by an enzyme immunoassay.

As shown in Table 3, only 1 clone among the separated 116 clones in the non-separation pool had an antibody production amount of 0.5 to 1.0 mg/L, while 4 antibody-producing clones which produced high concentrations of over 10.0 mg/L were selected in the secondary separation pool which had undergone the selection process using fluorescence activated cell sorter.

TABLE 3

| Antibody production | Non-separation | Secondary separation |
|---|---|---|
| reference (mg/L) | pool | pool |
| No more than 0.5 | 115/116 cells | 33/116 cells |
| Greater than 0.5 and | 1/116 cells | 1/116 cells |
| no more than 1.0 | | |
| Greater than 1.0 and | 0/116 cells | 28/116 cells |
| no more than 5.0 | | |
| Greater than 5.0 and | 0/116 cells | 50/116 cells |
| no more than 10.0 | | |
| Greater than 10.0 | 0/116 cells | 4/116 cells |

### Example 7

### Verification of Correlation Between Antibody-Producing Animal Cells and Animal Cells in which Fluorescent Protein is Simultaneously Expressed to Form Reassembled GFP

In order to verify the correlation between the degree of reassembled GFP expression and the production amount of the antibody, the degree of GFP expression and the specific antibody productivity were measured for the 30 clones separated individually by performing fluorescence activated cell sorting and an enzyme immunoassay. The degree of GFP expression using the fluorescence activated cell sorting was measured by first injecting each clone into a T-25 flask at a concentration of 0.7×10⁵ cells/mL, culturing each clone for 3 days at 37° C., further culturing each clone for 2 days at 30° C. to induce reassembly of GFP, and then measuring a GFP mean value through the fluorescence activated cell sorting. The specific antibody productivity through the enzyme immunoassay was measured by injecting each clone into a T-25 flask at a concentration of 0.7×10⁵ cells/mL, measuring each total number of surviving cells on the 2^{nd} and 4^{th} days of culturing, measuring the produced antibody through an enzyme immunoassay, and then measuring specific productivity. The correlation between the specific antibody productivity and the GFP mean value for each clone was plotted on a graph.

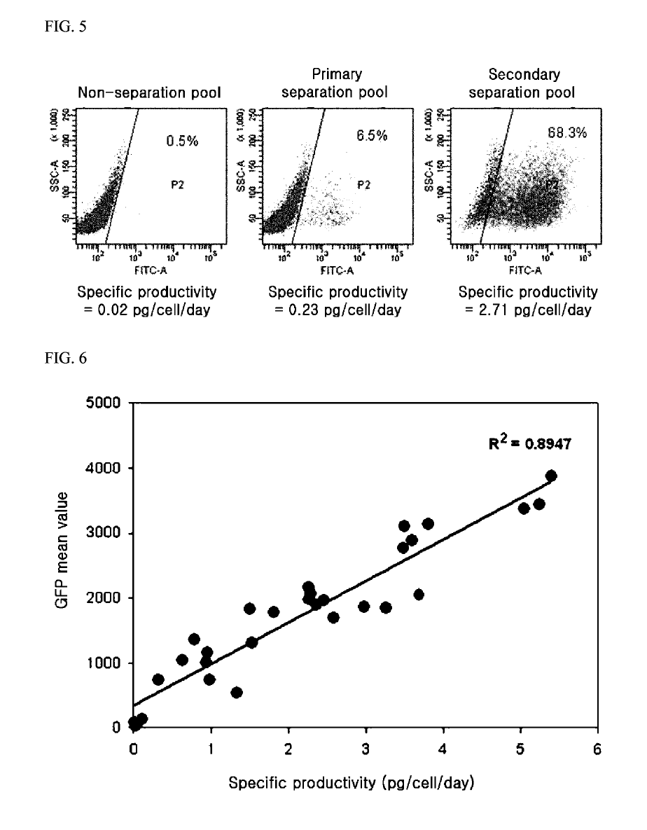

As a result, as shown in FIG. 6, the close correlation between the specific antibody productivity and the GFP mean value was shown, and as such, it was confirmed that GFP was a useful marker in selecting high-concentration antibody-producing cell lines.

While the invention has been shown and described with reference to certain exemplary embodiments thereof, it will be understood by those skilled in the art that various changes in form and details may be made therein without departing from the scope of the invention as defined by the appended claims.

What is claimed is:

1. A method for selecting antibody-producing cell lines, comprising:
transfecting a first expression vector and a second expression vector into cells, wherein the first expression vector comprises a sequence which encodes a first fragment of a fluorescent protein and a sequence which encodes a heavy chain of an antibody, and the second expression vector comprises a sequence which encodes a second fragment of the fluorescent protein and a sequence which encodes a light chain of the antibody; and
selecting the antibody-producing cell lines by confirming fluorescence derived from reassembly between the first fragment and the second fragment of the fluorescent protein.

2. The method according to claim 1, wherein the fluorescent protein is a green fluorescent protein (GFP), a red fluorescent protein (RFP), a blue fluorescent protein (BFP), a yellow fluorescent protein (YFP), a cyan fluorescent protein (CFP), or an enhanced fluorescent protein (EFP).

3. The method according to claim 1, wherein one of the first fragment and the second fragment of the fluorescent protein is a C-terminal fragment of the fluorescent protein, and the other is an N-terminal fragment of the fluorescent protein.

4. The method according to claim 1, wherein the first expression vector further comprises a sequence which encodes a first linker peptide conjugated to the sequence which encodes the first fragment of the fluorescent protein,
the second expression vector further comprises a sequence which encodes a second linker peptide conjugated to the sequence which encodes the second fragment of the fluorescent protein, and
the first linker peptide and the second linker peptide are constructed so as to be conjugated to each other.

5. The method according to claim 1, further comprising internal ribosome entry site (IRES) sequences between entry regions between the sequence which encodes the first fragment of the fluorescent protein of the first expression vector and an insertion site into which the sequence which encodes the heavy chain of the antibody are inserted; and between the sequence which encodes the second fragment of the fluorescent protein of the second expression vector and an insertion site into which the sequence which encodes the light chain of the antibody are inserted.

6. The method according to claim 1, further comprising:
confirming production of the antibody by the selected antibody-producing cell lines.

7. A kit for selecting antibody-producing cell lines, comprising:
a first expression vector comprising a sequence which encodes a first fragment of a fluorescent protein and an insertion site into which a sequence which encodes a heavy chain of an antibody may be introduced; and
a second expression vector comprising a sequence which encodes a second fragment of the fluorescent protein and an insertion site into which a sequence which encodes a light chain of the antibody may be introduced.


SEQUENCE LISTING (20 sequences)

SEQ ID NO: 1 - DNA, 18 bp, Artificial Sequence
  F1 Primer
atggtgagcaagggcgag

SEQ ID NO: 2 - DNA, 18 bp, Artificial Sequence
  R1 Primer
ctgcttgtcggccatgat

SEQ ID NO: 3 - DNA, 37 bp, Artificial Sequence
  F2 Primer
gcgaagcttgccaccatggtgagcaagggcgaggagc

SEQ ID NO: 4 - DNA, 35 bp, Artificial Sequence
  R2 Primer
gcggcggccgctcatggacccttcagcaaactggg

SEQ ID NO: 5 - DNA, 474 bp, Artificial Sequence
  Seq-1 DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcag

SEQ ID NO: 6 - DNA, 196 bp, Artificial Sequence
  Seq-2 DNA
ctacaacagccacaacgtctatatcatggccgacaagcagggtggctctggctctggctcgagcaagtctccagaagaactgaagggcattttcgaaaaatatgcagccaaagaaggtgatccaaaccaactgtccaaggaggagctgaagctactgcttcagacggaattccccagtttgctgaagggtccatga

SEQ ID NO: 7 - DNA, 18 bp, Artificial Sequence
  F3 primer
aagaacggcatcaaggtg

SEQ ID NO: 8 - DNA, 18 bp, Artificial Sequence
  R3 primer
tcagtacagctcgtccat

SEQ ID NO: 9 - DNA, 43 bp, Artificial Sequence
  F4 primer
gcgaagcttgccaccatgagcaccctcgatgagctttttgaag

SEQ ID NO: 10 - DNA, 33 bp, Artificial Sequence
  R4 primer
gcggcggccgctcagtacagctcgtccatgccg

SEQ ID NO: 11 - DNA, 243 bp, Artificial Sequence
  Seq-3 DNA
aagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtactga

SEQ ID NO: 12 - DNA, 154 bp, Artificial Sequence
  Seq-4 DNA
agcaccctcgatgagctttttgaagaactagacaagaatggagatggagaagttagtttcgaagaattccaggtgttggtgaaaaagatatcccagacgtcgggtggaagcggtaagaacggcatcaaggtgaacttcaagatccgccacaaca

SEQ ID NO: 13 - DNA, 33 bp, Artificial Sequence
  F5 primer
ctagctagcgccaccatgggatggagctatatc

SEQ ID NO: 14 - DNA, 26 bp, Artificial Sequence
  R5 primer
cgacgcgtctaacactctcccctgtt

SEQ ID NO: 15 - DNA, 33 bp, Artificial Sequence
  F6 primer
tgctctagagccaccatggtgagcaagggcgag

SEQ ID NO: 16 - DNA, 28 bp, Artificial Sequence
  R6 primer
gcgtgtcgactcatggacccttcagcaa

SEQ ID NO: 17 - DNA, 33 bp, Artificial Sequence
  F7 primer
ccggaattcgccaccatgggatggagctatatc

SEQ ID NO: 18 - DNA, 27 bp, Artificial Sequence
  R7 primer
ccggaattctcatttacccggagacag

SEQ ID NO: 19 - DNA, 33 bp, Artificial Sequence
  F8 primer
tgctctagagccaccatgagcaccctcgatgag

SEQ ID NO: 20 - DNA, 28 bp, Artificial Sequence
  R8 primer
gcgtgtcgactcagtacagctcgtccat